(19) United States
(12) Patent Application Publication
(10) Pub. No.: US 2026/0183137 A1
(43) Pub. Date: Jul. 2, 2026

(54) A CATHETER ASSEMBLY

(71) Applicant: ConvaTec Limited, Flintshire (GB)

(72) Inventor: Oliver Pfleger, Wirral (GB)

(21) Appl. No.: 19/535,087

(22) Filed: Feb. 10, 2026

Related U.S. Application Data
(63) Continuation of application No. PCT/GB2024/052104, filed on Aug. 9, 2024.
(60) Provisional application No. 63/518,608, filed on Aug. 10, 2023.

(30) Foreign Application Priority Data
Sep. 8, 2023 (GB) 2313721.9

(51) Int. Cl. A61F 5/453; A61M 25/00

(52) U.S. Cl. CPC A61F 5/453; A61M 25/002; A61M 2207/00

(57) ABSTRACT

A packaged catheter assembly comprises a pouch and a catheter contained within the pouch. The pouch comprises a fold line and the catheter is arranged across the fold line. The catheter assembly is movable between a folded configuration and an unfolded configuration, wherein in the folded configuration the pouch is folded about the fold line and the catheter is arranged in a coiled configuration. The pouch may be configured to provide an opening to allow the catheter to be withdrawn and separated from the pouch. The catheter may be arranged in a figure of eight when in the unfolded configuration. The catheter may cross the fold line at least twice. An angle between the catheter and the fold line may be less than 75 degrees. The fold line may divide the pouch into two equal sized square pockets.

### TECHNICAL FIELD OF THE INVENTION

The present invention relates to packaged catheter assemblies. In particular the invention concerns packaged urinary catheter assemblies, and most particularly, but not exclusively packaged intermittent male urinary catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

It is desirable for catheter assemblies to be compact so that they are easily portable. However, this can cause problems during use of the catheter as the components of the assembly are made smaller or are concealed within the assembly in a way that is difficult to access. This can make use of the catheter in a safe and controlled manner difficult. It can also result in damage to the catheter if it is stored in an inappropriate manner.

An example of an existing packaged catheter assembly is disclosed in US2014/0142555A1. This discloses reducing the size of the assembly by providing packaging that is foldable around the catheter. However, the size of the assembly is still restricted by the catheter itself; in particular, the length of the package is longer than that of the catheter itself.

It is an object of embodiments of the present invention to at least partially overcome or alleviate the above problems and/or to provide an improved packaged catheter assembly.

### SUMMARY OF THE INVENTION

In broad terms, the present invention concerns a catheter assembly comprising a catheter. The catheter assembly may comprise a pouch. The pouch may be configured to contain the catheter within it. The catheter may be contained within the pouch. The catheter assembly may therefore be a packaged catheter assembly. The pouch may comprise a fold line. The catheter may be arranged across the fold line. The catheter assembly may be movable between a folded configuration and an unfolded configuration. In the folded configuration, the pouch may be folded about the fold line. In the folded configuration, the catheter may be arranged in a coiled configuration.

According to a first aspect of the invention there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration.

In the coiled configuration, the catheter thereby defines an arc of at least 360 degrees. Advantageously, the coiling of the catheter when the catheter assembly is folded protects the catheter from damage as it ensures the catheter is not excessively curved, or folded. This allows the packaged catheter assembly to adopt a more compact and convenient form for the user to carry with them while retaining the full functionality of the catheter.

As the catheter assembly is moveable between the folded and unfolded configurations, this also provides the user with more flexibility in how they use the device. For example, a user with more limited dexterity may wish to maintain the assembly in the unfolded state to make it easier to access the catheter, whilst a user with better dexterity, but a smaller pocket bag to keep the catheter assembly in, may wish to fold the catheter assembly into the smaller folded configuration for transport.

The catheter may be a urinary catheter. The catheter may be a male urinary catheter. The catheter may be a female urinary catheter. The catheter may be an intermittent catheter. In one embodiment, the catheter is an intermittent male urinary catheter. Thus, the features of the present invention allow intermittent male urinary catheters to be used more easily and effectively given they are typically quite long and inconvenient to carry day-to-day.

Preferably, the pouch is sealed. Most preferably, the pouch provides a sterile seal. The packaged catheter assembly may therefore be a sealed packaged catheter assembly. Most preferably a sterile sealed packaged catheter assembly. This helps ensure the contents remains sterile before use.

An angle between the catheter and the fold line at the fold line may be less than 90 degrees, less than 75 degrees, less than 60 degrees or less than 45 degrees. Preferably, the angle between the catheter and the fold line at the fold line is less than 45 degrees. As the catheter is angled with respect to the fold line as mentioned above, this helps reduce the risk of the catheter being folded, during folding of the pouch, in such a way which could damage the catheter.

Accordingly in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein an angle between the catheter and the fold line at the fold line is less than 75 degrees or preferably, wherein the angle between the catheter and the fold line at the fold line is less than 45 degrees.

An angle between the catheter and the fold line at the fold line in the unfolded configuration may be greater than when in the folded configuration. In the unfolded configuration, the angle between the catheter and the fold line at the fold line may be less than 90 degrees, less than 75 degrees, less than 60 degrees or less than 45 degrees. Preferably, in the unfolded configuration, the angle between the catheter and the fold line at the fold line is less than 45 degrees. In the folded configuration, the angle between the catheter and the fold line at the fold line may be less than 45 degrees, less than 30 degrees, or less than 15 degrees. Preferably, in the folded configuration, the catheter and the fold line at the fold line are substantially parallel. The catheter may be arranged along the fold line. Thus, the catheter is arranged across the fold line but may also be arranged along it, this allows the catheter to curve along the length of the fold line without having to follow the sharp curvature of the pouch at the fold line.

In the coiled configuration, the catheter may define an arc of at least 420 degrees, at least 480 degrees, at least 540 degrees, at least 600 degrees, at least 660 degrees or at least 720 degrees. Preferably, in the coiled configuration, the catheter defines an arc of about 720 degrees. Thus, the catheter is efficiently packaged into a smaller space by coiling it up. With an arc over an increasing amount of degrees improves the ability of the catheter to remain in the same place within the pouch as it can be arranged to better interlock with itself, such as in a figure of eight arrangement described below, and also bears more effectively against the edges of the pouch without causing movement of the catheter inside the pouch.

In the coiled configuration, the catheter may be arranged on either side of the fold. In the coiled configuration, substantially half of the catheter may be arranged on either side of the fold. Thus, the catheter is more evenly distributed about the fold and is able to better remain in the coiled configuration.

In the coiled configuration, the catheter may be any suitable coiled shape, such as circular, elliptically shaped, or another shape like rectangular with rounded corners to avoid excessive curvature and damage to the catheter. Preferably, in the coiled configuration the catheter is substantially circular. Thus, the size of the packaged catheter assembly in any one direction is minimised, making it more compact overall.

The catheter may cross the fold line at least once. The catheter may cross the fold line at least twice. Preferably, the catheter crosses the fold line only twice. By crossing the fold line twice, the size of the packaging is further reduced compared to the length of the catheter.

Accordingly in one example, there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the catheter crosses the fold line at least twice.

The pouch may be configured to move the catheter into the coiled configuration when folded about the fold line. In the unfolded configuration, the catheter may not be arranged in a coiled configuration. Alternatively, in the unfolded configuration, the catheter may be arranged in a coiled configuration. In the unfolded configuration, the catheter may comprise at least two opposing turns along its length. In the unfolded configuration, the catheter may be arranged in a figure of eight. This allows the catheter to be spaced evenly across the available space within the pouch in the unfolded configuration so that it is easier to access.

Accordingly in one example, there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein in the unfolded configuration, the catheter is arranged substantially in a figure of eight.

In the unfolded configuration, a section of the catheter may curve in different directions either side of the fold line. In the folded configuration, a section of the catheter may curve in the same direction either side of the fold line. Preferably, a section of the catheter curves in different directions either side of the fold line, and in the folded configuration, curves in the same direction either side of the fold line. In the folded configuration, the catheter may comprise fewer opposing turns, or changes in turn direction, along its length than when in the unfolded configuration. In the folded configuration, the catheter may define a substantially continuous turn that has a longer arc length than when in the unfolded configuration. In the unfolded configuration, a section of the catheter arranged across the fold line may be substantially S-shaped. In the folded configuration, a section of the catheter arranged across the fold line may be substantially U-shaped or circular. Preferably, a section of the catheter is substantially S-shaped in the unfolded configuration and substantially U-shaped or circular in the folded configuration. In the unfolded configuration, the catheter may have a length longer than a height of the pouch. The height of the pouch may be defined perpendicular to the fold line. The length of the catheter may be defined as the distance along the catheter from its proximal end to its distal end. The catheter may be about twice as long as the height of the pouch when in the unfolded configuration. Thus, the catheter is more compactly stored.

Accordingly in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein in the unfolded configuration, the catheter has a length longer than a height of the pouch.

The pouch (or catheter assembly) may be provided in the unfolded configuration. The pouch may be foldable into the folded configuration. Thus, the pouch is provided to the user in a way that is more easy to handle and use, but can be folded to make the packaging more discreet and compact if required.

Alternatively, the pouch (or catheter assembly) may be provided in the folded configuration. This can provide a more compact package for the user at the point of sale or delivery without requiring them to manually fold it first.

The catheter may comprise a proximal end for insertion into the body and a distal end. The catheter may comprise a flexible tube connecting the proximal and distal ends. The distal end may comprise a funnel. The funnel may be configured to direct the flow of fluid out of the distal end. The funnel may provide a handling element for the catheter. This makes the catheter easier to use in embodiments where the catheter is removable/separatable from the pouch for use as an “open” catheter assembly where liquid flows out of the catheter and into a suitable receptacle such as a toilet.

Thus, in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the distal end of the catheter comprises a funnel configured to direct the flow of fluid out of the distal end of the catheter and optionally provide a handling element for the catheter.

The distal end of the catheter may be provided with a handling sleeve. The sleeve may be: no more than 25%; no more than 20%; no more than 15%; no more than 10%; or no more than 5% of the length of the catheter, but could be longer, even up to the full length of the catheter. The handling sleeve may be tubular. The handling sleeve may have a diameter that is larger than the diameter of the catheter. The handling sleeve may comprise a flexible plastics material. Embodiments of the present invention provide a funnel and sleeve to allow easy handling of the catheter.

The pouch may comprise a flexible plastics material. Preferably, the pouch is constructed of flexible sheets or films of plastic material. For example, polypropylene (PP), polyethylene terephthalate (PET), low density polyethylene (LDPE), metalized polyester (MET PET), polyvinyl chloride (PVC) or orientated polypropylene (OPP).

The pouch may comprise two walls. The pouch may comprise a peripheral seal between the two walls. The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. The peripheral seal may provide a sterile seal. Thus the peripheral seal can be easily constructed to suit the specific needs of the pouch and ensure the contents remains sterile within the pouch. Advantageously, additional protection is provided to ensure the catheter remains sterile.

The peripheral seal may form one or more edges of the pouch. In the unfolded configuration, the peripheral seal may form all the edges of the pouch. In the folded configuration, the fold line may form an edge of the pouch.

The two walls may comprise a front wall and a rear wall. The pouch may be configured to be folded about the fold line with the front wall on the inside of the fold. The peripheral seal may comprise a left lateral edge. The peripheral seal may comprise a right lateral edge. The peripheral seal may comprise an upper edge. The peripheral seal may comprise a base. The left and right lateral edges may be defined as the left and right edges when viewing the catheter assembly in the unfolded configuration with the front wall in front of the rear wall, the base at the bottom and the upper edge at the top. The pouch may comprise a substantially open internal volume. The interior of the pouch may be bounded by the edges defined by the peripheral seal, preferably the upper edge, left lateral edge, base and right lateral edge. The interior of the pouch may be substantially free of obstructions. This can allow the wetting fluid to more easily access the pouch and the catheter to be more easily withdrawn.

The pouch may have a width defined as the distance between the left lateral edge and right lateral edge of the pouch when in the unfolded configuration. The pouch may have a width of at least 80 mm, at least 100 mm, at least 120 mm, at least 140 mm, at least 160 mm or at least 180 mm. The pouch may have a width of no more than 250 mm, no more than 200 mm, no more than 160 mm, or no more than 120 mm. The pouch may have a height defined as the distance between the upper edge and base when in the unfolded configuration. The pouch may have a height of at least 80 mm, at least 100 mm, at least 120 mm, at least 140 mm, at least 160 mm or at least 180 mm. The pouch may have a height of no more than 250 mm, no more than 200 mm, no more than 160 mm, or no more than 120 mm. Preferably, the width is between 80-100 mm, and the height is between 160-200 mm. The pouch may have a height that is over double its width. Preferably, one of the width and height of the pouch is at least 50% greater than the other. This provides a longer thinner unfolded catheter assembly that can then be folded into a squarer shape that is convenient to be carried around by the user and more conducive to the coiled catheter being contained within it.

In the unfolded configuration, the pouch may have a height that is no more than four times its width. Preferably, the pouch may have a height no more than three times its width. The height of the pouch may be defined perpendicular to the fold line. The width of the pouch may be defined parallel to the fold line. This limits the amount of folds required to effectively reduce the size of the pouch.

Accordingly in one embodiment there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein, in the unfolded configuration, the pouch has a height of no more than four times its width, wherein the height of the pouch is defined perpendicular to the fold line.

The fold line may span completely between opposing sides of the pouch. The fold line may be provided across the width of the pouch. Where the fold line is provided across the width of the pouch, the fold line may be arranged towards a midpoint of the height of the pouch. The fold line may be provided across the height of the pouch. Where the fold line is provided across the height of the pouch, the fold line may be arranged substantially towards a midpoint of the width of the pouch. The fold line may be provided across the shorter of the width or height of the pouch. Thus, the fold line allows the longer of the width or height to be made shorter by folding the pouch about the fold line.

The fold line may be denoted by a marker on the outside of the pouch, for example on the front wall and/or rear wall of the pouch. The marker may be detectable on the outside of the pouch when the pouch is in the unfolded configuration. The marker may only be detectable when the pouch is in the unfolded configuration. This makes the pouch more discreet when folded. The marker may comprise a visible line across the pouch, for example a dashed line. The marker may be a different colour or shade of colour than the rest of the pouch. The marker may be a different texture or shape than the rest of the pouch. Thus, the user can identify where they should fold the pouch which reduces the likelihood that folding the pouch will damage the catheter.

Accordingly in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the fold line is denoted by a marker on the outside of the pouch.

The fold line may be configured to facilitate preferential folding of the pouch at the fold line. The fold line may comprise a region of different mechanical properties to the rest of the pouch, for example different mechanical properties to the walls of the pouch adjacent to the fold line. The pouch may comprise a groove or line of weakness which is more flexible than the rest of the pouch. Thus, the pouch can be more easily folded at the correct place.

When in the folded configuration,. The pockets may be formed from the two walls. The pockets may join to each other at the fold. The pockets may comprise the lateral edges of the pouch. Preferably, both pockets comprise both lateral edges of the pouch. The pockets may comprise either the upper edge or base of the pouch. In the folded configuration, one wall may form an exterior surface of the pouch, for example the rear wall. Preferably, in the folded configuration, the wall on the outside of the fold forms an exterior surface of the pouch, for example the rear wall. In the folded configuration, one wall may form a divider between the pockets. Preferably, in the folded configuration the wall on the inside of the fold forms a divider between the pockets, for example the front wall. The two pockets may be about the same size. The two pockets may be about the same shape. The catheter may extend across the fold from one pocket to the other. The proportion of a length of the catheter arranged in each pocket may be substantially the same. The proportion of the catheter arranged in each pocket may be 35-65% of the length of the catheter, or 40-60% of the length of the catheter, or about half of the length of the catheter. Thus, the compact folded configuration is enabled by the formation of two pockets.

Accordingly in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein, when in the folded configuration, the pouch comprises two pockets defined either side of the fold, wherein a proportion of a length of catheter arranged in each pocket is substantially the same.

Each pocket may be any suitable shape, but is preferably rectangular or most preferably approximately square. The aspect ratio (width:length) of each pocket may be 6:1 to 1:6, or 5:1 to 1:5, or 4:1 to 1:4, or 3:1 to 1:3, or 2:1 to 1:2, or 1.5:1 to 1:1.5, or about 1:1. The width/length of a pocket may be defined in the same direction as the width/height of the pouch. Thus, the pocket shape is controlled to ensure the catheter can be at least partly coiled in any pocket without damage.

Accordingly in one embodiment, there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein in the folded configuration, the pouch comprises two pockets defined either side of the fold, wherein each pocket has an aspect ratio (width:length) of 4:1 to 1:4.

The pouch may be configured to provide an opening to allow access to the contents of the pouch. The catheter may be configured to be withdrawn through the opening and preferably separated from the pouch. The opening may be provided in, or near, an edge of the pouch. Preferably, in or near the upper edge of the peripheral seal. The opening may be provided in only one of the two walls of the pouch. Preferably, the opening is provided in the front wall of the pouch. The opening may be suitable for repackaging of the catheter into the pouch through the opening. The opening may be sized to permit repackaging of the catheter into the pouch. The opening may have a width of at least 10% the length of the catheter, or at least 15% the length of the catheter, or about 20% the length of the catheter. The width of the opening being defined as a distance between two opposite sides of the opening, preferably in a direction parallel to the fold line. Thus, the opening can allow easy access to the catheter and enables the catheter to be easily repackaged if necessary, such as after use to hygienically store the used catheter before disposal.

Accordingly, in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the pouch is configured to provide an opening to allow access to the contents of the pouch, wherein the catheter is configured to be withdrawn through the opening and separated from the pouch, preferably wherein the opening is suitable for repackaging of the catheter into the pouch through the opening.

The opening may be provided separate from the peripheral seal. The opening may be separated from the peripheral seal. This can provide space for the sealing flap to be attached as mentioned below.

The opening may have a height measured parallel to the height of the pouch. The opening may have a height of at least 4%, at least 6% or at least 8% the height of the pouch. The opening may have a height of no more than 10%, no more than 8% or no more than 6% the height of the pouch. Preferably, the opening has a height of about 7% the height of the pouch. The opening may have a width measured parallel to the width of the pouch. The opening may have a width of at least 70%, at least 80% or at least 90% the width of the pouch. The opening may have a width of no more than 100%, no more than 90% or no more than 80% the width of the pouch. Preferably, the opening has a width of about 90% the width of the pouch. The opening may be any suitable shape or size, such as circular, elliptical, rectangular or irregularly shaped. Preferably, the opening is rectangular with rounded corners.

The distal end of the catheter may be arranged adjacent to the opening. The funnel may be arranged adjacent to the opening. The distal end or funnel of the catheter may be arranged centrally with respect to the opening. Thus, the user can gain access to the pouch and easily access the catheter by its distal end reducing the risk of compromising the sterility of the part of the catheter that enters the body such as the proximal end of the catheter.

The proximal end of the catheter may be arranged in the pouch at or towards one side of the opening. The proximal end of the catheter may be arranged in the pouch adjacent to the left or right lateral edge of the pouch. The proximal end of the catheter may not be arranged in the pouch towards a centre of the opening. Thus, the proximal end of the catheter is less likely to be touched by the user during opening the pouch which may compromise its sterility.

The proximal end and distal end of the catheter may be arranged on the same side of the fold line as one another. In the coiled configuration, the proximal and distal ends of the catheter may be arranged in the same pocket of the pouch. A midpoint along the catheter tube may be arranged on a different side of the fold line as the proximal and/or distal end of the catheter. A midpoint along the catheter tube may be arranged towards a base of the pouch. In the coiled configuration, the proximal and distal ends of the catheter may be arranged in a different pocket of the pouch from a midpoint along the catheter tube. This enables a more compact coiled shape.

The catheter tube may extend from the distal end of the catheter to the left (or right) lateral edge of the pouch. The catheter tube may then extend down across the fold line. The catheter tube may then extend to the right (or left) lateral edge of the pouch. The catheter tube may then turn back along the base of the pouch. The catheter tube may then extend to the left (or right) lateral edge of the pouch. The catheter tube may then extend back up across the fold line. The catheter tube may then extend to the proximal end of the catheter. The proximal end of the catheter may be positioned adjacent to the right (or left) lateral edge of the pouch. The above arrangement of the catheter in the pouch is preferably implemented when the catheter assembly is in the unfolded configuration. Thus, the catheter is arranged in a suitable figure of eight shape when in the unfolded configuration.

The opening may be substantially perpendicular to the fold line. This can provide better access to the pouch in the unfolded configuration.

The opening may be provided substantially parallel to the fold line. This can help provide an opening which is accessible even if the pouch is folded and which is more difficult for dirt to access. Accordingly in one example there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the pouch is configured to provide an opening to allow access to the contents of the pouch, wherein the catheter is configured to be withdrawn through the opening and separated from the pouch and the opening is substantially parallel to the fold line.

The fold line and the opening may be provided in different halves of the pouch. Where the fold line is provided across the width of the pouch, the fold line and opening may be separated by a distance of more than half the height of the pouch. Where the fold line is provided across the height of the pouch, the fold line and opening may be separated by a distance of more than half the width of the pouch. When the catheter assembly is in the folded configuration, the opening may be provided along an edge of the pouch distal from the fold line. Thus, the opening projects beyond the edge of the folded part of the pouch as the fold line is offset with respect to the middle of the pouch.

The pouch may comprise one or more fold lines. The pouch may comprise two or more fold lines. The fold line described above and herein may be a first fold line. As such, it will be understood that each reference to “the fold line” above may be substituted with reference to the first fold line, where there are first and second (or indeed any further) fold lines. The pouch may comprise a second fold line. The second fold line may be parallel to the first fold line. The first and second fold lines may be provided in different halves of the pouch. The second fold line may span between the left and right lateral edges of the pouch. The second fold line may be arranged closer to the upper edge of the pouch than the first fold line. The second fold line may be arranged no less than 10%, no less than 15%, no less than 20% or no less than 25% of the way from the upper edge to the base of the pouch. The second fold line may be arranged no more than 30%, no more than 25%, no more than 20% or no more than 15% of the way from the upper edge to the base of the pouch. Preferably, the second fold line is about 20% of the way from the upper edge to the base of the pouch. The first fold line may be arranged at a position approximately midway between the second fold line and the base of the pouch. In the folded configuration, the pouch may be folded at both the first and second fold lines. The pouch may be configured to be folded about the second fold line with the front wall on the inside of the fold. The pouch may be configured to be folded about the first fold line before being folded about the second fold line. The second fold line may be configured to move a portion of the pouch to overlie the rear wall of the pouch. Thus, the second fold line can allow a part of the pouch to be folded over on top of the already folded pouch to make the pouch more compact and intuitive to use.

The pouch may comprise a fastening element. The fastening element may be configured to retain the catheter assembly in the folded configuration. The fastening element may comprise any suitable re-sealable or temporary adhesive means such as a button, buckle, hook-and-hook or hook-and-loop fasteners, chemical adhesives, magnetic fasteners, etc. The fastening element may be a sticker. The fastening element may be configured to secure together the two edges of the pouch distal from the fold line. The fastening element may be configured to attach two parts of the rear wall to one another. Thus, the fastening element ensures the pouch is simply and easily secured in the folded configuration if desired.

The fastening element may be provided on the pouch. Where the catheter assembly is provided in the folded configuration, the fastening element may be provided on the pouch in a position to retain it in the folded configuration. Where the catheter assembly is provided in the unfolded configuration, the fastening element may be provided on the pouch. Due to the configuration of a fold line and the catheter arranged preferably at an angle to it, the catheter assembly is suitable for being retained in the folded state for extended periods of time without damage to the catheter.

Accordingly in one example, there is provided a packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein the pouch is provided in the folded configuration and optionally further comprising a fastening element provided on the pouch in a position to retain the pouch in the folded configuration.

The pouch may comprise an interaction region. The interaction region may facilitate user access to the contents of the pouch. Actuation of the interaction region may create, and/or provide access to, the opening in the pouch. Thus, the opening may be pre-formed in the pouch or may be formable, such as by actuation of the interaction region. The interaction region may be configured to tear upon actuation to form the opening. Where the opening is pre-formed it may be sealed closed until actuation of the interaction region. The pouch may comprise a breakable region, for example a part of the peripheral seal. The interaction region may comprise the breakable region of the peripheral seal. The interaction region may be provided adjacent the upper edge of the peripheral seal. The interaction region may be defined between the upper edge of the pouch and the second fold line. Thus, the pouch can be easily opened using the interaction region.

The interaction region may be concealed in the folded configuration. The interaction region may be on the inside of a fold of the pouch in the folded configuration. Thus, the interaction region is better protected from accidental damage or opening.

The breakable region may comprise the opening covered with a sealing flap. The sealing flap may be shaped to correspond to the shape of the opening. The sealing flap may be sealed to the pouch around a periphery of the opening by a flap seal. The flap seal may comprise a permanent edge configured to permanently attach the sealing flap to the pouch. The flap seal may comprise a breakable edge configured to temporarily attach a portion of the sealing flap to the pouch. The breakable edge is preferably configured to resealably attach to the pouch. Thus the sealing flap provides a convenient and secure means of sealing the opening and allowing access to the pouch.

The sealing flap may not extend beyond the flap seal along the permanent edge. Thus, the user is less likely to try and inadvertently break the flap seal along the permanently sealed edge.

The permanent edge may be adjacent to or along an edge of the peripheral seal. The permanent edge is preferably adjacent to or along the upper edge of the peripheral seal. The permanent edge may be positioned between the opening and the edge of the peripheral seal the permanent edge is adjacent to or along. This allows the user to grasp the pouch and then pull/tear across the relevant edge of the peripheral seal away from the pouch to open the pouch. This makes it easier to open.

The breakable edge may be provided around one or more edges of the flap seal. The breakable edge is preferably provided around all edges of the flap seal which do not comprise the permanent edge. The breakable seal may provide a sterile seal. The breakable seal and permanent edge may together provide a sterile seal.

Gaps in the flap seal may be provided between the breakable edge and the permanent edge. A gap may be provided between the respective ends of the permanent edge and the breakable edge. Each gap may be no more than 10%, no more than 8% or no more than 6% the height of the sealing flap. Each gap may be at least 1%, at least 2% or at least 5% the height of the sealing flap. Thus, the breakable edge and gaps ensure the sealing flap can be effectively rotated/moved out of the way of the opening.

The catheter assembly may comprise a fluid reservoir. The fluid reservoir may be configured to retain a wetting fluid within it. The fluid reservoir may be configured to release wetting fluid to activate the catheter. The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter. The fluid reservoir may be configured to retain at least 5, at least 10, at least 15, at least 25, or at least 50 ml of wetting fluid.

The fluid reservoir may be positioned within the pouch. The fold line may define two portions of the pouch either side of the fold. Where there are two or more fold lines, there may be three or more portions defined by the two or more fold lines. The fluid reservoir may positioned in the same portion as the proximal end of the catheter. The fluid reservoir and proximal end of the catheter may not be separated by a fold line. Thus, the fluid reservoir is positioned to better wet the catheter. Alternatively, the fluid reservoir may be positioned in a different portion of the pouch as the proximal end of the catheter. This may be preferred where the proximal end of the catheter is drawn through the portion of the pouch containing the fluid reservoir during withdrawal from the pouch.

The fluid reservoir may be positioned towards or adjacent a base of the pouch. The base may be distal from the opening. Thus, the fluid reservoir can be easier to locate and fluid released by the fluid reservoir is less likely to be leaked out of the opening.

The portions of the pouch may correspond to the pockets of the pouch.

The proximal end of the catheter may be positioned between the first and second fold lines. The proximal end of the catheter may be positioned adjacent to the second fold line.

The fluid reservoir may be positioned centrally within a portion of the pouch. The fluid reservoir may be positioned between the first and second fold lines, preferably midway between the first and second fold lines. The fluid reservoir may be jammed inside the pouch. The fluid reservoir may span substantially the whole way between the left and right lateral edges of the pouch. This can help to ensure the fluid reservoir does not move inside the pouch and is less likely to be accidentally removed from the pouch.

The fluid reservoir may be positioned in between the opening and a majority of the length of the catheter. The fluid reservoir may be positioned with no more than one fold line between the fluid reservoir and the opening. The second fold line may separate the fluid reservoir and the opening. Preferably, the second fold line is the only fold line between the fluid reservoir and the opening. Thus, the fluid reservoir is positioned to ensure the catheter is adequately wetted as it is withdrawn through the pouch and out of the opening.

The fluid reservoir may comprise a deformable, frangible or burstable sachet. The outside of the pouch may comprise an activation marker configured to indicate the location of the fluid reservoir inside the pouch. This helps the user to release wetting fluid from the fluid reservoir before opening the pouch which helps ensure the catheter is adequately wetted before use and reduces spillages of wetting fluid.

The activation marker may be provided on a wall of the pouch, preferably the rear wall. The activation marker may be provided on the fastening element. The fastening element may comprise visual indicia thereon to indicate to the user how to release fluid from the fluid reservoir, for example the word “PRESS”. An activation marker may also be provided on the fluid reservoir. Thus, the catheter assembly is intuitive to use.

The invention also concerns a pack comprising a plurality of packaged catheter assemblies. Each of the plurality of packaged catheter assemblies may be a packaged catheter assembly of the first aspect or as described in broad terms herein, and may each comprise any one or more of the features of a catheter assembly described above. The plurality of packaged catheter assemblies may be provided in the pack in the unfolded configuration.

Accordingly, in a second aspect of the present invention there is provided a pack comprising a plurality of packaged catheter assemblies according to the first aspect of the invention, wherein each of the plurality of packaged catheter assemblies are provided in the pack in the unfolded configuration.

Accordingly, the second aspect provides a pack comprising a plurality of packaged catheter assemblies, wherein each of the plurality of packaged catheter assemblies comprises a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration, wherein each of the plurality of packaged catheter assemblies are provided in the pack in the unfolded configuration

Advantageously, the catheter assemblies are provided unfolded which makes them easier to handle and can allow closer packing of them in the pack. The user can then fold each assembly if required.

Each of the plurality of packaged catheter assemblies may individually comprise any one or more of the optional features of the packaged catheter assembly of the first aspect. Each of the plurality of packaged catheter assemblies may, or may not, include the same optional features of the packaged catheter assembly of the first aspect of the invention as one another.

The pack may comprise a plurality of fastening elements. The plurality of fastening elements may be provided independent of the packaged catheter assemblies. Where the fastening elements are provided independently of the packaged catheter assemblies they may be provided with a release liner. Two or more fastening elements may be provided on the same release liner. Thus, the user can selectively use the fastening elements on the catheter assemblies if they decide to fold the catheter assemblies. This also improves the sealing ability of the fastening elements as they have not been previously used.

According to a third aspect of the present invention there is provided a method of manufacturing a packaged catheter assembly, the method comprising providing a pouch comprising a fold line, providing a catheter and arranging the catheter in the pouch and across the fold line such that the catheter assembly is movable between a folded configuration and an unfolded configuration, wherein in the folded configuration the pouch is folded about the fold line and the catheter is arranged in a coiled configuration.

The method of the third aspect may be a method of manufacturing a packaged catheter assembly of the first aspect of the invention, which, of course, may include any optional feature outlined above.

The method may comprise arranging the catheter into a figure of eight.

The method may comprise checking the packaged catheter assembly is in the unfolded configuration. The method may comprise moving the packaged catheter assembly to the unfolded configuration if it is in the folded configuration. Thus, the catheter assembly is provided in the unfolded configuration.

The method may comprise manufacturing a plurality of packaged catheter assemblies. The method may comprise forming a pack comprising the plurality of packaged catheter assemblies. Each of the plurality of packaged catheter assemblies may be provided in the unfolded configuration.

According to a fourth aspect of the present invention there is provided a method of stowing a packaged catheter assembly, the packaged catheter assembly comprising a pouch and a catheter contained within the pouch, wherein the pouch comprises a fold line and the catheter is arranged across the fold line, wherein the method comprises moving the catheter assembly from an unfolded configuration to a folded configuration by folding the pouch about the fold line such that the catheter is arranged in a coiled configuration.

The packaged catheter assembly may be a packaged catheter assembly of the first aspect of the invention, which, of course, may include any optional feature outlined above, and may be manufactured according to the third aspect of the invention.

The method may comprise folding the catheter assembly about two fold lines. The method may comprise folding the catheter assembly about a first fold line and then a second fold line. The catheter may be arranged across the first fold line.

According to a fifth aspect of the present invention there is provided a method of unpackaging a packaged catheter assembly, the packaged catheter assembly comprising a pouch and a catheter contained within the pouch, wherein the pouch comprises a fold line and the catheter is arranged across the fold line, wherein the catheter assembly is in a folded configuration with the pouch folded about the fold line and the catheter is in a coiled configuration, the method comprising moving the catheter assembly from the folded configuration to an unfolded configuration by unfolding the pouch about the fold line, and forming an opening in the pouch to access its contents.

The packaged catheter assembly may be a packaged catheter assembly of the first aspect of the invention, which, of course, may include any optional feature outlined above, and may be manufactured according to the third aspect of the invention.

The method may include bursting a fluid reservoir contained within the pouch, preferably while the catheter assembly is in the folded configuration. The method may comprise locating an activation marker. The method may comprise pinching or pressing the pouch to release fluid from the fluid reservoir.

The method may include removing the catheter from the pouch. Preferably, an opening is formed while the catheter assembly is in the unfolded configuration.

The method may comprise introducing the catheter by its proximal end into the urethra. The method may comprise allowing fluid to pass out of the body through the catheter. Consequently, the method may be a method of using a catheter assembly.

The methods of the third to fifth aspects may include any feature, optional or otherwise, of one another.

### DETAILED DESCRIPTION OF THE INVENTION

In order that the invention may be more clearly understood one or more embodiments thereof will now be described, by way of example only, with reference to the accompanying drawings, of which:

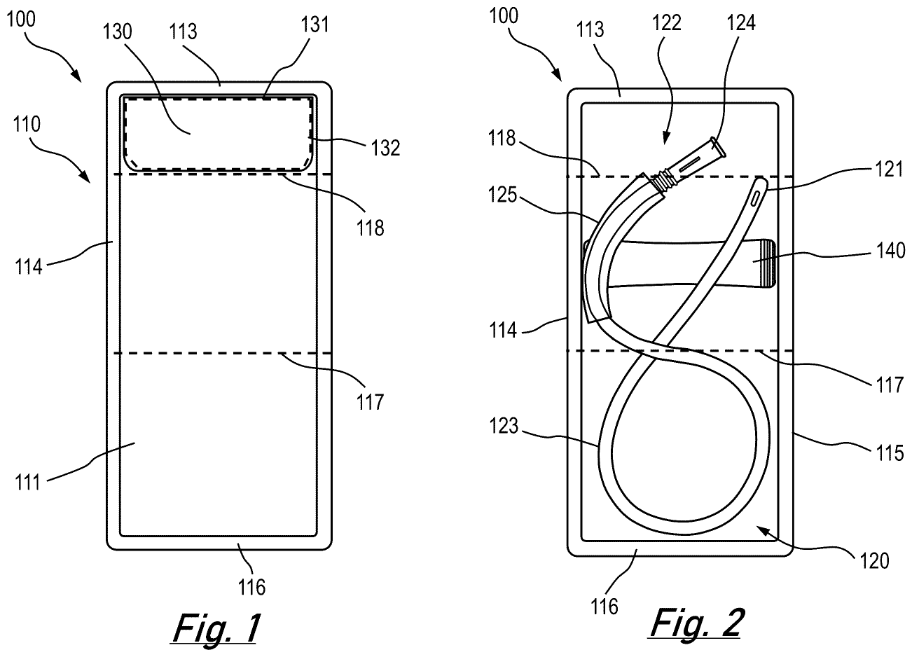

FIG. 1 is a front view of an embodiment of a packaged catheter assembly in the unfolded configuration;

FIG. 2 is a front view of the packaged catheter assembly of FIG. 1 showing the contents of the pouch;

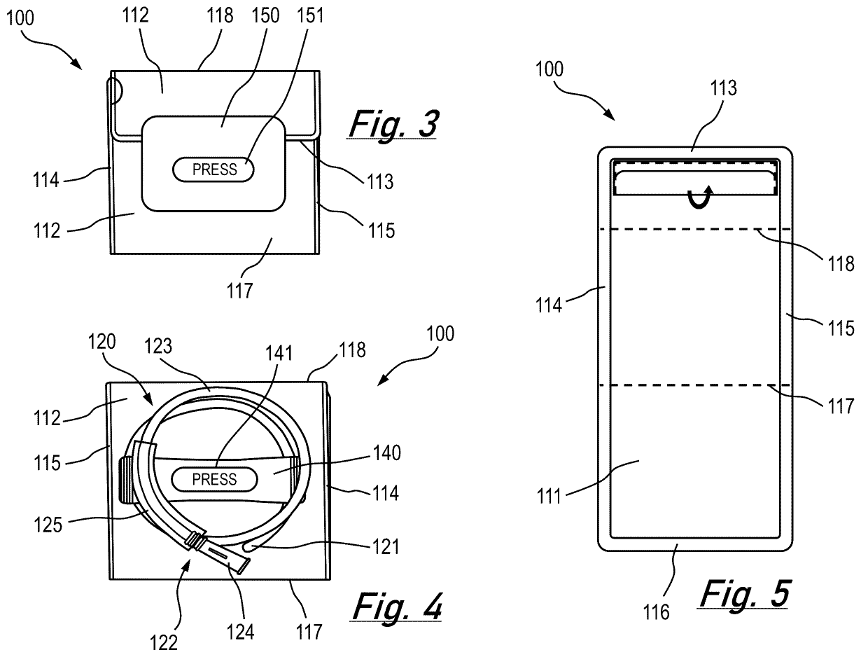

FIG. 3 is a front view of the packaged catheter assembly in the folded configuration;

FIG. 4 is a rear view of the packaged catheter assembly of FIG. 1 in the folded configuration showing the contents of the pouch;

FIG. 5 is a front view of the packaged catheter assembly of FIG. 1 during opening of the pouch;

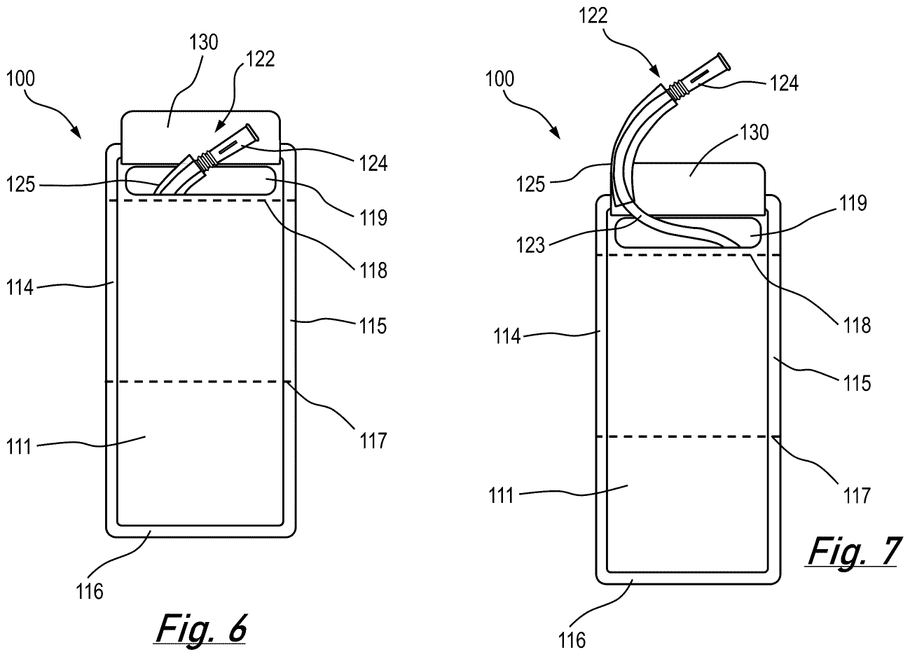

FIG. 6 is a front view of the packaged catheter assembly of FIG. 1 during removal of the catheter from the pouch;

FIG. 7 is a front view of the packaged catheter assembly of FIG. 1 during removal of the catheter from the pouch;

FIG. 8 is a cross-sectional view of the packaged catheter assembly of FIG. 1 in the unfolded configuration;

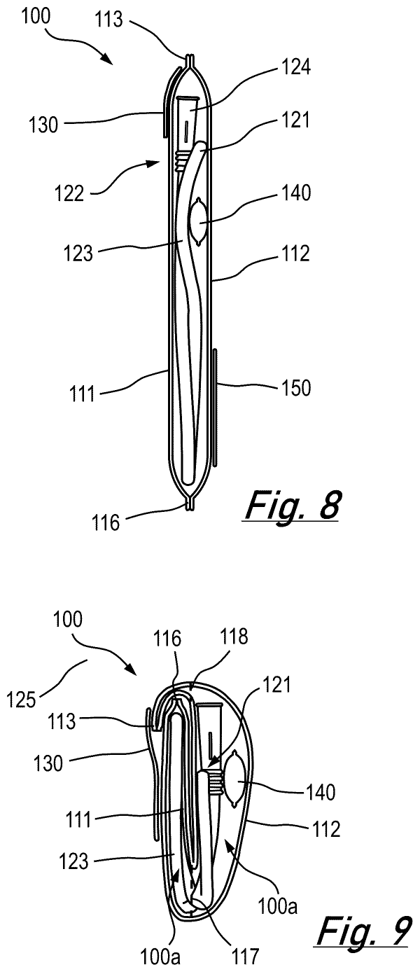

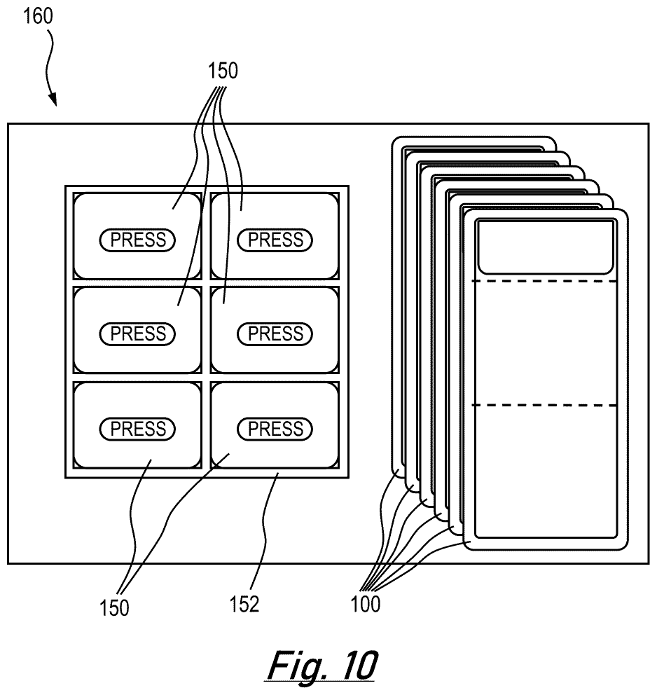

FIG. 9 is a cross-sectional view of the packaged catheter assembly of FIG. 1 in the folded configuration; and

FIG. 10 is a schematic diagram of an embodiment of a pack containing a plurality of packaged catheter assemblies.

Referring to FIGS. 1 to 9, an embodiment of a packaged catheter assembly 100 is shown. In this embodiment, the catheter assembly 100 comprises a pouch 110 and catheter 120. The catheter 120 has a proximal end 121 for insertion into the body and a distal end 122 connected by a flexible tube 123. In this embodiment, the catheter 120 is a male urinary catheter made from a hydrophilic thermoplastic elastomer (TPE). Obviously those skilled in the art will be able to select suitable alternative examples.

In this embodiment, the distal end 122 comprises a funnel 124 to guide urine from the bladder out of the tube 123 in use. A handling sleeve 125 (not shown in FIGS. 8 and 9) is also provided around the tube 123 adjacent to the distal end 122. In this embodiment, the catheter 120 has a length of between 30-50 cm, for example 40 cm, and the handling sleeve is between 10 and 30% the length of the catheter, for example 20-25%, e.g. 11 cm. In this embodiment, the catheter 120 is provided with a hydrophilic coating, this is activated when it contacts the fluid held in the fluid reservoir 130. In other embodiments, any suitable catheter length or type may be used.

In this embodiment, the pouch 110 comprises a front wall 111, a rear wall 112, and a peripheral seal joining the periphery of the walls 111, 112 together to form the pouch. In this embodiment, the front and rear walls 111, 112 are both opaque and formed of a flexible plastics film material such as polypropylene (PP), polyethylene terephthalate (PET), low density polyethylene (LDPE), metalized polyester (MET PET), polyvinyl chloride (PVC) or orientated polypropylene (OPP). The peripheral seal comprises any one or more of: a weld; mechanical seal; heat seal; pressure seal; adhesive; solvent bond; ultraviolet bond; ultrasonic weld; laser weld; impulse weld; or friction weld.

The pouch 110 further comprises a first fold line 117 and in this embodiment also includes an optional second fold line 118. The catheter assembly and pouch thereby have an unfolded configuration wherein the pouch is not folded about the fold lines 117, 118 and a folded configuration wherein the pouch is folded about the fold lines 117, 118.

In this embodiment, the first fold line 117 and second fold line 118 are denoted on the outside of the pouch 110 by markers comprising dashed lines of a different colour or shade of colour to the rest of the pouch 110. This helps to guide the user to fold the pouch 110 at the correct location to ensure the catheter 120 is not damaged by folding the pouch 110. In other embodiments, the pouch may comprise a tactile or mechanical feature to help the user locate the fold lines, or may comprise a mechanical feature in the pouch to facilitate preferential folding of the pouch at the fold lines, for example the pouch may comprise a groove or line of weakness which is more flexible than the rest of the pouch. The markers may only be detectable when the pouch is in the unfolded configuration to make the pouch more discreet when folded.

The peripheral seal forms the edges of the pouch when it is in the unfolded configuration and comprises a base 116, a left lateral edge 114, a right lateral edge 115, and an upper edge 113. The fold lines 117, 118 both span completely between the left and right lateral edges 114, 115 and are both parallel to the upper edge 113 and base 116. When the pouch is in the folded configuration the fold lines 117, 118 form some of the edges of the pouch as described below.

In this embodiment the left lateral edge 114 and right lateral edge 115 are defined as the left and right sides of the pouch when viewing the pouch 110 in the unfolded configuration with the rear wall 112 behind the front wall 111, the base 116 at the bottom of the pouch 110 and the upper edge 113 at the top of the pouch 110. The peripheral seal thus defines a pouch 110 that is generally rectangular and suitably has a width between the lateral edges of between 60-110 mm, for example about 80 mm, and a height from the base 116 to the upper edge 113 of between 100 to 250 mm, for example about 200 mm. When unfolded, the base 116 defines the bottom of the pouch in use, and the upper edge 113 the top. However, in other embodiments alternative shapes and sizes could be conceived, such as an elliptical or circular pouch.

In this embodiment, the second fold line 118 is positioned about 20% of the way from the upper edge 113 to the base 116. The first fold line 117 is positioned about midway between the second fold line 118 and the base 116. This allows the pouch to be folded about the first fold line 117 and then the second fold line 118 into a more compact shape.

In this embodiment, the pouch 110 comprises an interaction region to facilitate user access to the contents of the pouch 110. The interaction region comprises an opening 119 in the pouch 110 covered by a sealing flap 130. In this embodiment, the opening 119 is positioned near to the upper edge 113 on the front wall 111 and spaced from the peripheral seal. The opening 119 is generally rectangular with rounded corners, a width of about 90% the width of the pouch and a height of about 10% the width of the pouch. The width of the opening 119 is therefore about 20% the length of the catheter. The opening 119 thereby provides access to the contents of the pouch 111 as described below, and permits easy access to the catheter as well as repackaging of the catheter into the pouch if required after use.

In this embodiment, the sealing flap 130 extends from the upper edge 113 down to the second fold line 118. The sealing flap 130 corresponds to the shape of the opening 119 and extends substantially all the way between the left and right lateral edges 114, 115 of the pouch 110.

In this embodiment, as best seen in FIG. 1, the sealing flap 130 is sealed to the pouch 110 around the periphery of the opening 119 by a flap seal. The flap seal comprises a permanent edge 131 configured to permanently attach the sealing flap 130 to the pouch 110 and a breakable edge 132 configured to temporarily attach a portion of the sealing flap 130 to the pouch 110. The permanent edge 131 is provided along an edge of the sealing flap 130 adjacent to the upper edge 113, this helps ensure the sealing flap 130 does not become separated from the pouch 110 which would be inconvenient for the user.

In this embodiment, the breakable edge 132 is provided around the rest of the edge of the sealing flap 130 and is therefore generally U-shaped. The breakable edge 132 provides a sterile seal to ensure that the catheter 120 remains sterile prior to use.

In this embodiment, when the catheter assembly 100 is in the unfolded configuration, the catheter 120 is arranged in the pouch 110 in a rough figure of eight arrangement with the funnel 124 arranged centrally across the width of the pouch 110 and adjacent to the opening 119. The catheter tube 123 then extends to the left lateral edge 114 before curving back across and down the pouch 110 to the right lateral edge 115. The tube 123 crossing the first fold line 117 approximately midway between the left and right lateral edges 114, 115. The tube 123 then turns back along the base 116 to the left lateral edge 114 again, before crossing back over the first fold line 117. The proximal end 122 is arranged adjacent to the second fold line 118 but between the first and second fold lines 117, 118 and adjacent to the right lateral edge 115. Thus, the catheter 120 crosses the first fold line 117 twice. The magnitude of the angle between the first fold line 117 and the catheter 120 each time the catheter crosses the fold line are all within about 40 degrees of one another and are each about 45 degrees or less. Of course, the catheter 120 could be arranged a different way round, for example with the proximal end 121 adjacent to the left lateral edge 114 instead. This shape helps ensure the catheter 120 adopts a coiled configuration when the catheter assembly 100 is folded and makes the catheter assembly easier to fold as described below.

Due to the shape as described above, sections of the catheter 120 arranged over the first fold line 117 are also arranged in rough S-shapes when in the unfolded configuration. For example, an S-shape is formed between the funnel 124, across the first fold line 117 and down to the base 116. An S-shape is also formed from the base 116 up across the first fold line 117 and to the proximal end 121 of the catheter 120. The situation may also be understood by appreciating that the sections of the catheter across the fold line curve in different directions either side of the fold line when in the unfolded configuration.

In this embodiment, the catheter assembly 100 further comprises a fluid reservoir 140 containing wetting fluid and provided inside the pouch 110. The fluid reservoir 140 is an elongate rectangular burstable sachet of water, but in other embodiments any suitable reservoir may be used. In this embodiment, the fluid reservoir contains about 20 ml of water. In other embodiments, many other types of reservoir would be suitable including different shapes and sizes of reservoir, or non-burstable reservoirs and other external sources of fluid. In addition, other fluids could be used, for example lubricants, gels, oils or the like.

In this embodiment, the fluid reservoir 140 spans substantially completely between the left and right lateral edges 114, 115 and is thereby jammed in position such that it cannot easily move. In alternative embodiments, it could be glued or otherwise held in position. The fluid reservoir is positioned midway between the first and second fold lines 117, 118 and therefore in the same portion (or pocket 100a, see below) of the pouch 110 as the proximal end 121 of the catheter 120 and also between the opening and the majority of the length of the catheter 120. This helps to ensure that the proximal end 121 and other parts of the catheter are suitably wetted before use which reduces the risk of injury/discomfort to the user during use.

In this embodiment, the packaged catheter assembly 100 comprises a folded configuration in which it is folded about the first and second fold lines 117, 118. The catheter assembly 100 is preferably folded about the first fold line 117 with the front wall 111 on the inside of the fold and then the second fold line 118 also with the front wall 111 on the inside. The upper edge 113 and base 116 therefore no longer define the outer edges of the catheter assembly and instead these are defined by the second fold line 118 and first fold line 117 respectively.

In this embodiment, in the folded configuration, the catheter 120 adopts a coiled configuration as it defines an arc of at least 360 degrees. In this embodiment, due to the figure of eight shape in the unfolded configuration, the catheter 120 defines an arc of substantially 720 degrees. As such, upon folding, the opposing turns and changes of direction required to form the figure of eight in the unfolded configuration are effectively removed leaving a continuous coiled catheter with a single long turn one direction. This ensures the catheter 120 is compact and allows the size of the catheter assembly to be reduced without damaging the catheter.

In addition, in the folded configuration, the sections of the catheter 120 arranged over the first fold line 117 mentioned above are no longer S-shaped and now adopt U-shaped or circular configurations. In this embodiment, the sections adopt substantially circular configurations. Thus, the sections of the catheter either side of the fold line curve in the same direction as one another.

In this embodiment, in the folded configuration, the catheter 120 is substantially parallel to the first fold line 117 where it crosses the now folded first fold line 117. Due to the folding, the angle between the catheter 120 and the first fold line 117 at the fold line is reduced compared to the unfolded configuration, and in this embodiment the catheter 120 is substantially parallel to the first fold line 117 when crossing it. This allows the catheter to be curved around, across and along the fold without excessive curvature that may damage the catheter.

In this embodiment, in the folded configuration, the sealing flap 130 is hidden as it is on the inside of the fold created around the second fold line 118. It is thereby protected from accidental opening or damage before use.

In this embodiment, in the folded configuration, two pockets 100a are defined in the pouch 100 either side of the first fold line 117 and as shown best in FIG. 9. These pockets 100a are defined by the portions of the pouch 100 between the first and second fold lines 117, 118 and the first fold line 117 and the base 116 respectively. As the front wall 111 is on the inside of the fold about the first fold line 117, the front wall 111 forms a divider between the two pockets 100a and the rear wall 112 forms an exterior surface of the pouch 100. The two pockets 100a are rectangular and almost square due to the shape of the pouch and have an aspect ratio (width:length) of about 1:1. As described above, the catheter 120 extends across the fold from one pocket to the other in its coiled configuration. In this embodiment, the proportion of the catheter arranged in each pocket is the same, so about half the length of the catheter is arranged in each pocket.

In this embodiment, the catheter assembly 100 further comprises a fastening element in the form of a sticker 150 configured to retain the catheter assembly in the folded configuration. The sticker 150 is rectangular and comprises a resealable adhesive on one side that is configured to attach to the pouch to retain it in the folded configuration. In this embodiment, the sticker 150 is configured to attach to the rear wall 112 adjacent to the upper edge 113 and also to the rear wall 112 between the first fold line 117 and the base 116 to hold the catheter assembly 100 in the folded configuration.

In this embodiment, the sticker 150 further comprises an activation marker 151 in the form of visual indicia on the surface of the sticker to instruct the user how to release wetting fluid from the fluid reservoir 140. In this embodiment, the activation marker 151 comprises the word “PRESS” and is positioned at a point corresponding to the fluid reservoir 140 in the pouch. This encourages the user to release wetting fluid before removing the catheter 120 from the pouch 110.

In this embodiment, the fluid reservoir 140 also comprises a similar activation marker 141.

In this embodiment, the catheter assembly 100 is manufactured by providing the rear wall 112, catheter 120 and fluid reservoir 140 and arranging them on the rear wall 112 in the positions described above. The front wall 111 is then provided with the opening 119 pre-formed in it. The front wall 111 can then be placed over the rear wall 112 and the pouch formed with the contents inside by forming the peripheral seal around their periphery. This may be done in any suitable manner such as a weld; mechanical seal; heat seal; pressure seal; adhesive; solvent bond; ultraviolet bond; ultrasonic weld; laser weld; impulse weld; or friction weld.

In this embodiment, the sealing flap 130 is then attached to the pouch 110 by forming the permanent and breakable edges 131, 132.

In this embodiment, the packaged catheter assembly is provided to the user where it is provided in an unfolded configuration. Consequently, the sticker 150 is provided and stuck to the pouch on the rear wall 112. It can then be peeled back and re-stuck onto the pouch 110 to retain it in the folded configuration if required. The catheter assembly 100 is now ready to be provided to the user.

As shown in FIG. 2, in the unfolded configuration, the height of the pouch (defined perpendicular to the fold lines) is about half the total length of the catheter from its proximal end to distal end. Thus, the catheter is already stored in a compact manner even when unfolded, and folding further enhances this.

In a different embodiment, the catheter assembly may be provided in the folded configuration. In such embodiments, the catheter assembly is folded about the first fold line to move the base into alignment with the second fold line. The second fold line is then folded to move the sealing flap over the folded rear wall. The fastening element is then provided and stuck over the folded catheter assembly in the position described above to retain it in the folded configuration.

In this embodiment, to use the catheter assembly, where it is provided in the unfolded configuration, the user may decide to stow the catheter assembly so that it is more convenient to be carried around with them. They can do this by following the steps of folding and retaining as described above.

In this embodiment, the fluid reservoir 140 is preferably burst while the catheter assembly is in the folded configuration. This helps to ensure the wetting fluid is concentrated onto the proximal end 121 of the catheter 120. Consequently, the user may locate the activation marker 151 and pinch or press the pouch together to burst the fluid reservoir 140 and cause wetting fluid to be released into the pouch 110.

In this embodiment, the user can move the catheter assembly 100 to the unfolded configuration by removing the sticker 150 and unfolding the second fold line 118 and then first fold line 117. During the step of unfolding, the wetting fluid will then be able to more easily access the whole of the pouch 110 to more completely wet the catheter tube 123.

In this embodiment, the user can then access the contents of the pouch 110 by holding the pouch 110 in one hand and grasping the sealing flap 130 in the other. They can then pull their hands apart to break the breakable seal 132 and reveal the opening.

The funnel 124 is positioned adjacent to the opening and the user may grasp it and/or the handling sleeve 125 and withdraw the catheter 120 from the pouch 110. Advantageously, as the proximal end 121 is adjacent to a lateral edge of the pouch it is less likely to be touched by the user which may render it unsuitable for use.

Once the catheter 120 is removed from the pouch 110, the proximal end 121 may be progressively inserted into the body until fluid flows through the catheter 120 and out of the funnel. After use, the catheter 120 may be repackaged into the pouch 110 and disposed of.

Referring to FIG. 10, a pack 160 comprising a plurality of packaged catheter assemblies 100 is shown. Each of the plurality of packaged catheter assemblies 100 in the pack have many features in common with the packaged catheter assembly of FIGS. 1-9 described above, so like numerals are used and only differences are described.

The only difference between the individual packaged catheter assembly described above and each of the packaged catheter assemblies 100 of the pack 160 is that the sticker 150 is provided separately from the catheter assembly 100.

In this embodiment, there are six packaged catheter assemblies 100 and six stickers 150. The stickers 150 are provided together on a release liner 152 in a 3-by-2 arrangement. The release liner 152 protects the adhesive side of the stickers 150 until use and ensures they are effective at retaining the catheter assembly 100 in the folded configuration.

Preferably, the plurality of packaged catheter assemblies 100 are all provided in the unfolded configuration, this allows the pack to be more compact and also reduces unnecessary stresses on the catheters.

To manufacture the pack, the release liner 152 comprising stickers 150 is provided and the packaged catheter assemblies manufactured as described above. They are then combined together into a single pack, for example by placing them into a suitable packaging.

In use, the user may remove a single packaged catheter assembly and move it into the folded configuration as described above, then retain it in this configuration by taking a sticker 150 from the release liner 152 and attaching it to the pouch 110. The user can then carry this folded catheter assembly 100 around with them until it is needed, and use it as described above.

The one or more embodiments are described above by way of example only. Many variations are possible without departing from the scope of protection afforded by the appended claims. For example, the embodiment described includes both a first fold line, which the catheter crosses, and second fold line, and the invention envisages embodiments which only have the first fold line that is crossed by the catheter and no second fold line. Likewise, embodiments are envisaged with more than two fold lines.

1. A packaged catheter assembly comprising a pouch and a catheter contained within the pouch, wherein the pouch comprises a fold line and the catheter is arranged across the fold line, wherein the catheter assembly is movable between a folded configuration and an unfolded configuration, wherein in the folded configuration the pouch is folded about the fold line and the catheter is arranged in a coiled configuration.

2. A catheter assembly according to claim 1 wherein the catheter is an intermittent male urinary catheter.

3. A catheter assembly according to claim 1 or claim 2 wherein the distal end of the catheter comprises a funnel configured to direct the flow of fluid out of the distal end of the catheter and optionally provide a handling element for the catheter.

4. A catheter assembly according to any one of the preceding claims wherein the pouch is configured to provide an opening to allow access to the contents of the pouch, wherein the catheter is configured to be withdrawn through the opening and separated from the pouch.

5. A catheter assembly according to claim 4 wherein the opening is suitable for repackaging of the catheter into the pouch through the opening.

6. A catheter assembly according to claim 4 or claim 5 wherein the opening is substantially parallel to the fold line.

7. A catheter assembly according to claim 6 wherein the fold line and opening are provided in different halves of the pouch.

8. A catheter assembly according to any one of the preceding claims wherein, when in the folded configuration, the pouch comprises two pockets defined either side of the fold, wherein each pocket has an aspect ratio (width:length) of 4:1 to 1:4.

9. A catheter assembly according to any one of the preceding claims wherein, in the unfolded configuration, the pouch has a height of no more than four times a width of the pouch, wherein the height of the pouch is defined perpendicular to the fold line and the width of the pouch is defined parallel to the fold line.

10. A catheter assembly according to any one of the preceding claims wherein, when in the folded configuration, the pouch comprises two pockets defined either side of the fold, wherein a proportion of a length of catheter arranged in each pocket is substantially the same.

11. A catheter assembly according to any one of the preceding claims wherein an angle between the catheter and the fold line at the fold line is less than 75 degrees.

12. A catheter assembly according to claim 10 wherein the angle between the catheter and the fold line at the fold line is less than 45 degrees.

13. A catheter assembly according to any one of the preceding claims wherein in the coiled configuration, the catheter defines an arc of at least 540 degrees.

14. A catheter assembly according to any one of the preceding claims wherein in the coiled configuration, the catheter is substantially circular.

15. A catheter assembly according to any one of the preceding claims wherein the catheter crosses the fold line at least twice.

16. A catheter assembly according to any one of the preceding claims wherein in the unfolded configuration, a section of the catheter curves in different directions either side of the fold line and in the folded configuration curves in the same direction either side of the fold line.

17. A catheter assembly according to any one of the preceding claims wherein in the unfolded configuration, the catheter is arranged substantially in a figure of eight.

18. A catheter assembly according to any one of the preceding claims wherein the fold line is denoted by a marker on the outside of the pouch.

19. A catheter assembly according to any one of the preceding claims wherein in the unfolded configuration, the catheter has a length longer than a height of the pouch.

20. A catheter assembly according to any one of the preceding claims wherein in the folded configuration, the pouch comprises two pockets either side of the fold and the catheter extends across the fold from one pocket to the other.

21. A catheter assembly according to any one of the preceding claims wherein the pouch is provided in the unfolded configuration and is foldable into the folded configuration.

22. A catheter assembly according to any one of claims 1 to 20 wherein the pouch is provided in the folded configuration.

23. A catheter assembly according to claim 22 further comprising a fastening element provided on the pouch in a position to retain the pouch in the folded configuration.

24. A pack comprising a plurality of packaged catheter assemblies according to any one of the preceding claims, wherein each of the plurality of packaged catheter assemblies are provided in the pack in the unfolded configuration.

25. A method of manufacturing a packaged catheter assembly, the method comprising providing a pouch comprising a fold line, providing a catheter and arranging the catheter in the pouch and across the fold line such that the catheter assembly is movable between a folded configuration and an unfolded configuration, wherein in the folded configuration the pouch is folded about the fold line and the catheter is arranged in a coiled configuration.

26. A method of unpackaging a packaged catheter assembly, the packaged catheter assembly comprising a pouch and a catheter contained within the pouch, wherein the pouch comprises a fold line and the catheter is arranged across the fold line, wherein the catheter assembly is in a folded configuration with the pouch folded about the fold line and the catheter is in a coiled configuration, the method comprising moving the catheter assembly from the folded configuration to an unfolded configuration by unfolding the pouch about the fold line, and forming an opening in the pouch to access its contents.

27. A method according to claims 25 or 26 wherein the packaged catheter assembly is the catheter assembly of any of claims 1 to 23.
